(19) United States
(12) Patent Application Publication
(10) Pub. No.: US 2014/0043939 A1
(43) Pub. Date: Feb. 13, 2014

(54) IMAGING BY EXTRAPOLATION OF VECTOR-ACOUSTIC DATA

(71) Applicant: Ivan Vasconcelos, Coton (GB)

(72) Inventor: Ivan Vasconcelos, Coton (GB)

(73) Assignee: WESTERNGECO L.L.C., HOUSTON, TX (US)

(21) Appl. No.: 14/112,891

(22) PCT Filed: May 18, 2012
(86) PCT No.: PCT/IB12/00972

§ 371 Date: Oct. 18, 2013

Related U.S. Application Data
(60) Provisional application No. 61/489,425, filed on May 24, 2011.

(51) Int. Cl. G01V 1/28; G01N 29/26; A61B 8/00

(52) U.S. Cl. CPC G01V 1/282; A61B 8/00; G01N 29/26; G01V 2210/675
USPC 367/73

(57) ABSTRACT

Methods for wavefield extrapolation using measurements of a wavefield quantity and a component of the gradient of the wavefield quantity are disclosed. The methods use “exact” representations of scattering reciprocity. The methods can yield “exact”, nonlinear, “true-amplitude” receiver wavefields that are beyond the receiver measurement boundary. Methods of evaluating/validating the extrapolated data are also disclosed. Some methods may also evaluate the accuracy of models for the areas where data are extrapolated or measured. These methods can be used in any industries involving imaging, such as geophysical/seismic exploration, bio-medical imaging, non-destructive remote sensing, acoustic space architecture, design and engineering.

### CROSS-REFERENCE TO RELATED APPLICATIONS

This application claims the benefit of U.S. Provisional Application No. 61/489,425 filed on May 24, 2011 and titled of “Exact Inverse Receiver Extrapolation of Vector-Acoustic data” by the same inventor, the disclosure of which is incorporated by reference herein in its entirety.

### BACKGROUND

This disclosure relates to extrapolation of vector-acoustic wavefield data for acquiring data that otherwise cannot be acquired, where the data may be needed in various fields, such as geophysical exploration, medical imaging, engineering and construction.

Data extrapolation or interpolation may be used in the situation when data cannot be directly acquired or it is not reasonable/feasible to directly collect the data. In technologies where wave phenomenon/properties are being acquired, wavefield extrapolation may be used to indirectly determine the data.

### SUMMARY

This summary is provided to introduce a selection of concepts that are further described below in the detailed description. This summary is not intended to identify key or essential features of the claimed subject matter, nor is it intended to be used to limit the scope of the claimed subject matter.

This disclosure describes methods of depth extrapolation (also known as “wavefield redatuming in depth”) that use measurements of a physical quantity (e.g. pressure) and one or more components of its gradient (which components may be in any form, e.g., pure gradient, or particle displacement/velocity/acceleration). The methods use exact representations of scattering reciprocity. The extrapolated data may be used to yield exact, nonlinear, “true-amplitude” receiver wavefields and these “true-amplitude” receiver wavefields can then be used for vector-acoustic imaging techniques, which are widely used in many fields involving imaging, such as geophysical/seismic exploration, bio-medical imaging, non-destructive remote sensing, acoustic space architecture, design and engineering. In addition, methods to measure the accuracy of the extrapolated data and the underlying object models are also described.

### BRIEF DESCRIPTION OF DRAWINGS

Embodiments of this disclosure are described with reference to the following figures. The same numbers are used throughout the figures to reference like features and components.

FIG. 1 illustrates a general data acquisition plan implementing methods disclosed in this application, where the object in interest is entirely outside the sensor boundary.

FIG. 2 illustrates several different source-receiver configurations in accordance with embodiments of the present invention, where FIG. 2a illustrates a configuration for a monopole source and FIG. 2b illustrates a configuration for a dipole source.

FIG. 3 illustrates a step in a method for wavefield extrapolation, in accordance with one embodiment of the present invention.

FIG. 4 illustrates a step in a method for wavefield extrapolation, in accordance with one embodiment of the present invention.

FIG. 5 illustrates a plane-based acquisition plan, where two planes “enclose” the sensor boundary.

FIG. 6 illustrates a “single” plane acquisition plan, where a “free surface” is utilized.

FIGS. 7a and 7b illustrate application of wavefield extrapolation to a marine seismic survey in accordance with FIG. 6.

FIG. 8 illustrates a diagram where the wavefield extrapolation is applied to a marine seismic survey in accordance with FIG. 6, where the survey uses over and under receivers.

FIG. 9 illustrates a sample processing system that implements methods described in the current application.

FIG. 10 illustrates a flow diagram of a method for extrapolating data for an object that is outside the sensor boundary, in accordance with an embodiment of the present invention.

FIG. 11 illustrates a flow diagram of a method for extrapolating data for an object that is outside the sensor boundary with two models, in accordance with an embodiment of the present invention.

FIG. 12 illustrates a flow diagram of a method for extrapolating data with a convolution type extrapolation, in accordance with an embodiment of the present invention.

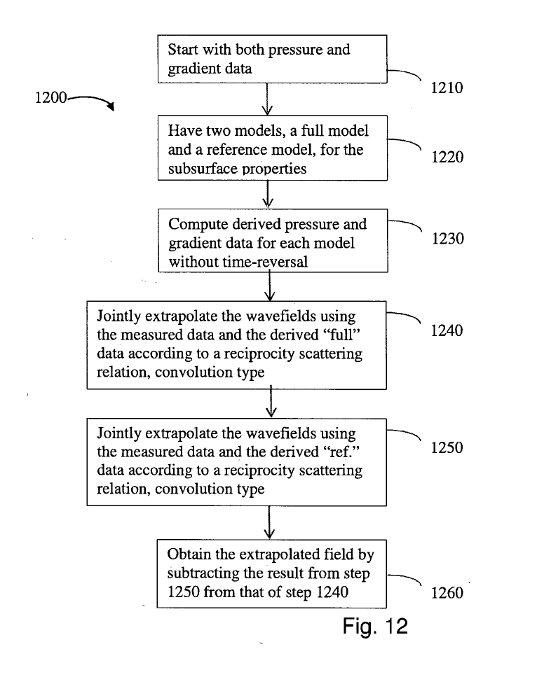

FIG. 13 illustrates a flow diagram of a method for computing an identity for evaluating the accuracy of a model, in accordance with an embodiment of the present invention.

FIG. 14 illustrates a flow diagram of a method for computing an identity for evaluating the accuracy of a model and/or an extrapolated wavefield, in accordance with an embodiment of the present invention.

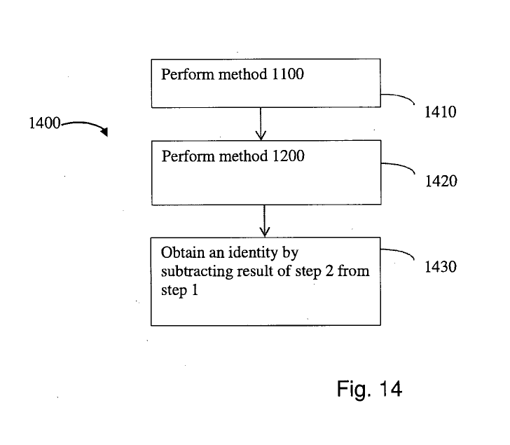

FIG. 15 illustrates a flow diagram of a method for computing an identity matrix for evaluating the accuracy of models relative to various model parameters, in accordance with an embodiment of the present invention.

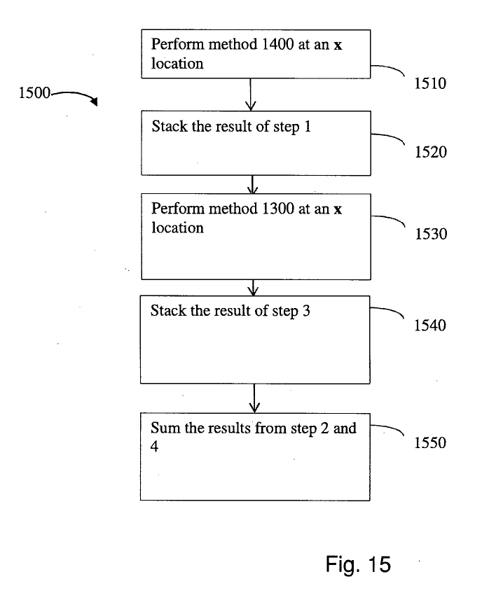

FIG. 16 illustrates a flow diagram of an indirect data acquisition method, in accordance with an embodiment of the present invention.

### DETAILED DESCRIPTION

Reference will now be made in detail to embodiments, examples of which are illustrated in the accompanying drawings and figures. In the following detailed description, numerous specific details are set forth in order to provide a thorough understanding of the subject matter herein. However, it will be apparent to one of ordinary skill in the art that the subject matter may be practiced without these specific details. In other instances, well-known methods, procedures, components, and systems have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step. The first object or step, and the second object or step, are both objects or steps, respectively, but they are not to be considered the same object or step.

The terminology used in the description of the disclosure herein is for the purpose of describing particular embodiments only and is not intended to be limiting of the subject matter. As used in this description and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

In this application, new methods of using wavefield extrapolation are disclosed. The methods can be used in any fields where wave phenomenon is involved and where not all relevant parts of the wavefield can be measured. The technical fields include at least: geophysical exploration such as seismic exploration, seismic imaging; Controlled Source Electromagnetic (CSEM) surveys; bio-medical imaging, for example, where ultrasound is used to construct image of internal body organs; construction and engineering where internal structure of an object is to be determined or where acoustic spaces is determined using active sound experiments. For simplicity, only examples in marine seismic survey are given to illustrate the implementation of the methods. They are for illustrative purposes only and not to limit the scope of the invention. In the application, the sources are the parts that emit signals. Receivers are the parts that receive signals. The sources and receivers are interchangeable, unless specifically stated otherwise. In this application, a monopole source is a source that generates scalar field, e.g. the common pressure sources, such as explosive discharge or an airgun etc. A dipole source is a source that is both a sink and a source in close proximity, e.g. a jet engine or a magnet. For a jet engine, in front of it, there is a negative pressure “source” (i.e. sink) and at the rear of it, there is positive pressure source. A magnet can generate a magnetic field. A monopole receiver is a receiver that measures a scalar quantity, such as a hydrophone. A dipole receiver is a receiver that measures at least one component of a vector quantity, such as a geophone measuring a vertical component of particle velocity of an elastic seismic wavefield, or a magnetometer measuring a horizontal component of magnetic field.

The wavefield quantity as discussed in this application can be any scalar physical quantity in a wavefield, such as for example a pressure in an acoustic wavefield, an intensity in a electromagnetic wavefield (light) and/or the like. The wavefield quantity may also be a component of a vector quantity, such as for example the vertical component of a particle velocity of a shear wavefield and/or the like. The gradient of the wavefield quantity is simply the vector field which points in the direction of the greatest rate of increase of the wavefield quantity (a scalar field) and whose magnitude is the greatest rate of change. As it is apparent in the discussion below, in some embodiments of the present invention, the measurement of the wavefield quantity and only one of the three components of its gradient are required for extrapolation. Although in most cases more measurements can make the methods work better, not all components of its gradient are needed. Examples are provided where the scalar wavefield quantity is a pressure in a seismic wavefield and/or a component of a vector quantity is, e.g., a vertical component of a particle velocity in an elastic seismic wavefield. However, the interpolation methods of the present invention may be used for analysis of different wavefields using different scalar wavefield quantities and/or vector quantities.

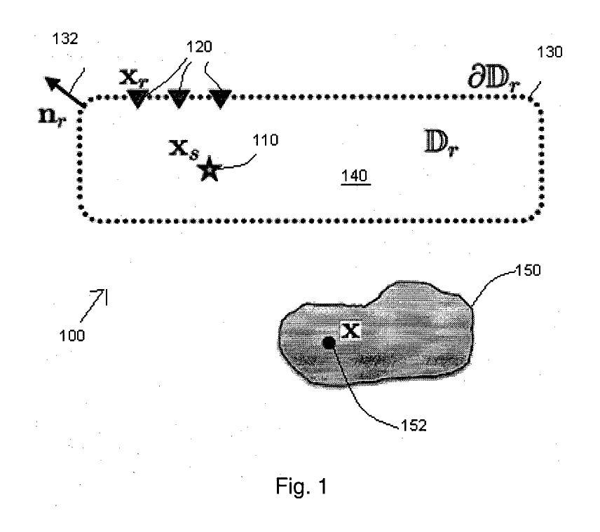

FIG. 1 depicts a general data acquisition plan 100 according to some of the methods of the present invention. An active source 110 at Xₛ as indicated with a star in FIG. 1 emits a signal, which is received by one or more receivers 120 at Xᵣ, as indicated with a triangle in FIG. 1. The signal received by receivers 120 at Xᵣ is influenced by an object 150, as indicated with gray shade, where a point of interest X 152 is located. It is desirable to find the wavefield or properties of the object 150 at any point X 152 using the source information at Xₛ and receiver information at Xᵣ. For any surveys, source 110 and receivers 120 are placed within a finite volume Dᵣ 140 enclosed by a boundary ∂Dᵣ. 130 where nᵣ 132 is the normal direction of the boundary at a location Xᵣ. If the object at X is within the boundary ∂Dᵣ or at least partially enclosed by the boundary, i.e. a receiver or source can be placed at the location X and a direct measurement made (not shown in FIG. 1), then the problem is not too difficult. Techniques like the one described above may be used for X-ray imaging, ultra-sound imaging and/or the like. However, the described method is not usable when X lies entirely outside the boundary 130, i.e. no direct measurement can be made, as shown in FIG. 1. Thus, other, “extrapolation” methods are needed for such situations.

In some embodiments of the present invention, nonlinear methods may be used for extracting the unknown scattered-wave response between the source 110 at Xₛ and a remote subsurface location X 152 (where no measurements are available), from the observed wavefield quantity (e.g. pressure and its gradient; a vertical component of particle velocity data and its gradient, on the receiver side) due to that same source, 110, which can be either a monopole source, a dipole source, or both kinds of sources, and receivers 120 Xᵣ on the enclosing boundary 130.

In the context of this general geometry as shown in FIG. 1, it is noted that the output extrapolation point X and all of the scatterers/reflectors/medium perturbations lie in a remote location 150 with respect to the acquisition experiment denoted by the enclosing receiver boundary ∂Dᵣ 130. This means that the exact extrapolated scattering response at a remote location 152 is obtained without the need for receivers 120 enclosing the subsurface target 152. Previous methods, such as the previous full-wave extrapolation methods, relied on receiver geometries that enclosed the targets for the obtained response to be exact. Given the joint information from a wavefield quantity and its gradient, the methods presented here extract remote scattering responses without the need of receivers 120 to enclose remote targets 152. The term “exact”, as used in this application, means that the retrieved response comprises all of the “true amplitude” arrivals, including all free-surface and internal multiple-scattered/reflected waves, as long as the subsurface model used for extrapolation is correct. The subsurface model may contain many physical, geophysical, stratigraphic or other related properties of the object which may or may not be directly measured.

Taking advantage of the geometry in FIG. 1 and the fact that pressure and gradient receivers are jointly available, the combination of both correlation- and convolution-type scattering reciprocity relations yields integral identities that can be used to determine whether or not the subsurface model used for extrapolation is adequate. These identities can be used to update the subsurface model for optimal receiver extrapolation. Also as an example, a practical marine seismic acquisition plan is presented and discussed below, which utilizes many methods disclosed here. This acquisition plan uses a modified geometry as in FIG. 1 to take advantage of the free-surface (i.e., the water surface).

Extrapolation

One embodiment of the present invention, utilizes the so-called one- and two-way reciprocity relations for scattered fields. (See Vasconcelos et al., “REPRESENTATION THEOREMS AND GREEN'S FUNCTION RETRIEVAL FOR SCATTERING IN ACOUSTIC MEDIA”, Phys. Rev. E 80, 036605 (2009), which is incorporated by reference for all purposes herein).

Combining the so-called one- and two-sided scattering reciprocity relations for the particular scattering configuration in FIG. 1 yields the following relation:

$${G}_{S} ( x , {x}_{s} ) = {∮}_{\partial {D}_{r}} \frac{- 1}{i ω ρ} [ {∇}_{{x}_{r}} {G}^{*} ( {x}_{r} , {x}_{s} ) {G}_{S} ( x , {x}_{r} ) - {G}^{*} ( {x}_{r} , {x}_{s} ) {∇}_{{x}_{r}} {G}_{S} ( x , {x}_{r} ) ] \cdot {n}_{r} {d}^{2} {x}_{r} \qquad Equation 1$$

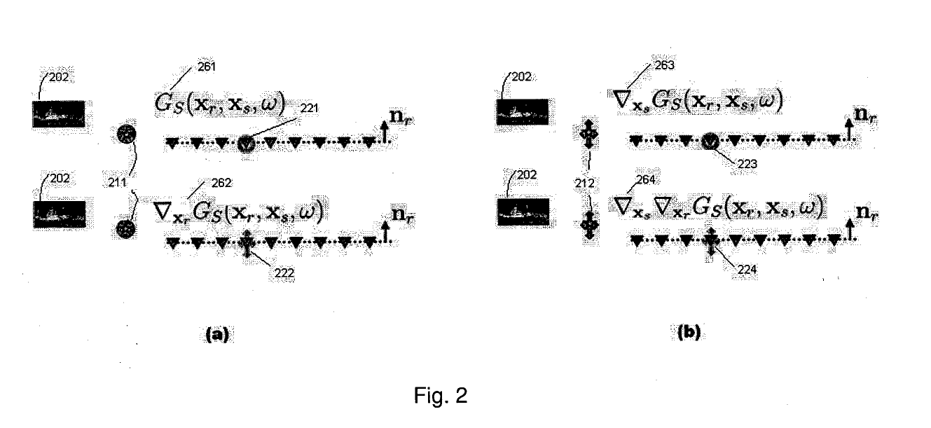

In Equation 1, all fields are assumed to be in the frequency domain although frequency dependence is omitted for brevity. (See FIG. 2 where frequency dependency is kept). It is noted that the use of frequency domain is for clarity and simplicity only. All methods discussed here' can be applied in the time domain with the same validity, as long as the proper time-frequency transformations are done. On the left-hand side (“LHS”) of Eq 1 is the desired extrapolated scattered receiver field G_{S}(x,xₛ), i.e. the wavefield at location X due to a source at Xₛ. On the right-hand side (“RHS”) of the Eq 1 is the extrapolation integral that uses the data G(xᵣ,xₛ) that are observed by receivers at Xᵣ due to physical sources at Xₛ (together with the model-derived extrapolator G_{S}(x,xᵣ) to predict the subsurface extrapolated receiver field. Eq 1 provides the exact extrapolated wavefield at X, based on acquired data G(xᵣ,xₛ) and a model-derived extrapolator G_{S}(x,xᵣ). The * superscript in Eq 1 denotes the complex-conjugate in the frequency domain, which translates to time-reversal in the time domain.

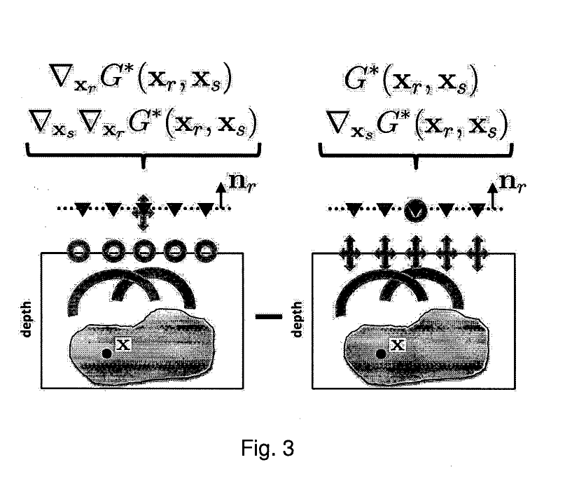

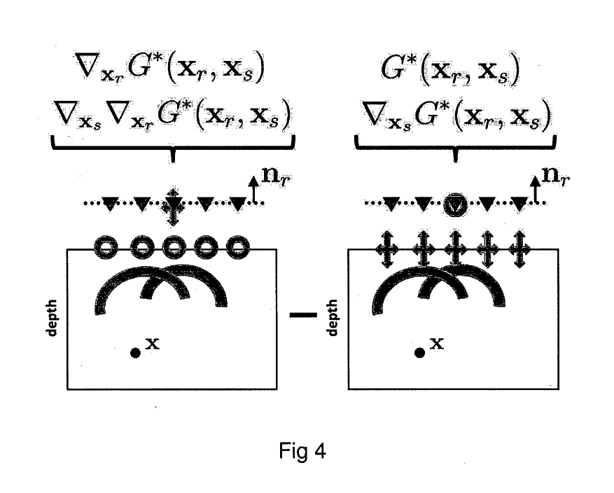

FIG. 2 is an exemplary illustration of several possible configurations for marine seismic data acquisition using: (a) a monopole-source 211; and (b) a dipole-source 212. The signals, excited by a physical source of either monopole type (a) 211 or (b) dipole type 212, are recorded by both monopole receivers 221 or 223 (e.g. hydrophones or similar pressure receivers, upper figures) and multicomponent gradient receivers 222 or 224 (e.g. accelerometers or the like, lower figures), which may be disposed in seismic streamers. Here, the G_{S} data represent scattered waves only, while G=G_{S}+G₀ in Eq 1 denotes the full data acquired at the receivers. G₀ is the wavefield without the scatterers. More discussion is presented in reference to FIGS. 3 and 4 below.

FIG. 2a illustrates the configurations with monopole sources 211. The upper illustration shows a monopole source 211 with monopole receivers 221. The lower illustration shows a monopole source 211 with dipole receivers 222 (e.g. geophones, accelerometers and/or the like).

FIG. 2b illustrates the configurations with dipole sources 212. The upper illustration shows a dipole source 212 with monopole receivers 223. The lower illustration shows a dipole source 212 with dipole receivers 224.

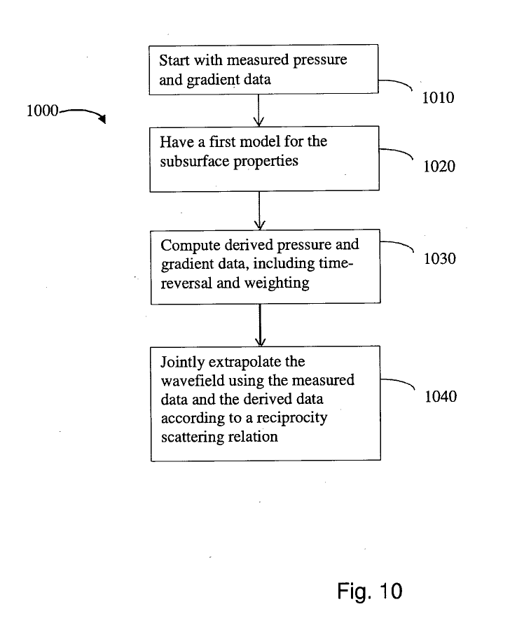

In reference to Eq 1 and FIG. 2 and FIG. 10, a method 1000 for extrapolation in accordance with one embodiment of the present invention may include the following steps:
start with both pressure G (xᵣ, xₛ) 261 and gradient data ∇ₓ_{ᵣ}G(xᵣ, xₛ) 262 on the receiver side (1010);
start with a known model for subsurface properties, and generate Gs(x, xᵣ) and its gradient ∇ₓ_{ᵣ}Gs(x,xᵣ) (1020);
time-reverse both pressure G*(xᵣ,xₛ) and gradient data ∇ₓ_{ᵣ}G*(xᵣ, xₛ);
weight data individually at receiver locations using the known (−iωρ(xᵣ))⁻¹ factor prior to either wavefield injection or convolution with extrapolators Gs, and project pressure and gradient data to local receiver normal direction nᵣ(xᵣ) according to Eq 1 (1030); and
jointly extrapolate time-reversed pressure and gradient receiver data using the model by evaluating the receiver integral (this yields the subsurface field Gs(x, xₛ) at all x locations within the subsurface model (1040)).

From a subsurface model, there are many ways to generate Gs(x, xᵣ) and its gradients. Sometimes, Gs(x, xᵣ) is referred to as a propagator. Methods to implement/generate the propagator Gs (x, xᵣ) include ray theory, finite difference, etc.

As mentioned above, the recorded data G is the total wavefield recorded at the receivers, which includes not only the desired scattering field G_{S}, but also other waves, such as direct arrivals, multiples etc. Another way to obtain the extrapolated wavefield Gs(x, xₛ) is to start with two models instead of one, where one of the models should contain all known heterogeneities/scatterers/perturbations (hereafter referred to as the “full model”) G, while the other model does not contain heterogeneities/scatterers/perturbations (hereafter referred to as the “reference model”) that account for the desired scattered field G₀. One way to make the reference model for this purpose is to “smooth” out the “full model” to derive the “reference model.” The heterogeneities/scatterers/perturbations in the full model, e.g. heterogeneities, sharp reflectivity/velocity contrasts are smoothed-out. The reference model contains the homogeneous properties of the object or the medium properties.

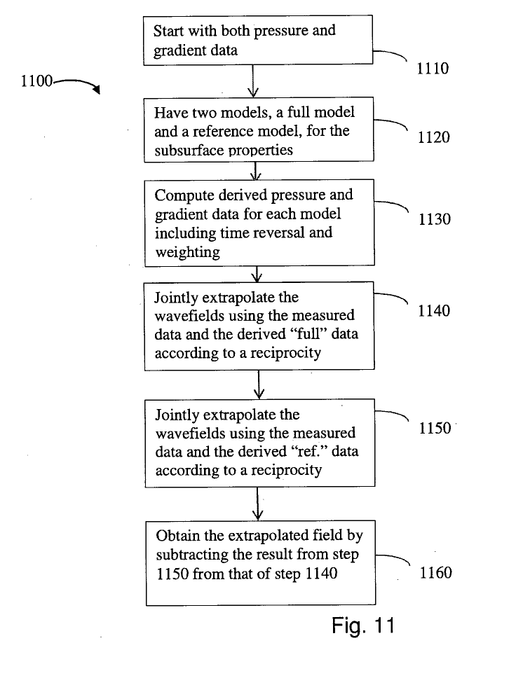

With the assistance of these two models (with reference to Eq 1, FIGS. 2, 3 and 4, and FIG. 11) a method 1100 for extrapolation in accordance with an embodiment of the present invention may include the following steps:
(1) start with both pressure 261 and gradient data 262 on the receiver side (see FIG. 2) (1110);
(2) start with 2 separate a priori, known models for subsurface proprieties, the “full model” G, and the “reference model” G₀ (1120);
(3) time-reverse both pressure and gradient data and weight data individually at receiver locations by the known (−iωρ(xᵣ))⁻¹ factor prior to wavefield injection or convolution with extrapolators, project pressure and gradient data to local receiver normal direction nᵣ(xᵣ) according to Eq 1 (1130);
(4) jointly extrapolate time-reversed pressure and gradient receiver data using the full model according to FIG. 3, by simultaneously using data from all receivers (this implicitly evaluates the receiver integral); this yields the subsurface field G(x,xᵣ) at all x locations within the subsurface model (1140);
(5) jointly extrapolate time-reversed pressure and gradient receiver data using the reference model according to FIG. 4, by simultaneously using data from all receivers (this implicitly evaluates the receiver integral); this yields the subsurface field G₀(x,xᵣ) at all x locations within the subsurface model (1150); and
(6) obtain the subsurface extrapolated field G_{S}(x,xᵣ) from Eq 1 by subtracting the result from step 5—(1150) G₀(x,xᵣ) from that of step 4—(1140) G(x,xᵣ) (1160).

FIG. 3 illustrates, in accordance with an aspect of the present invention, Step 5 of the method 1100, above, for receiver wavefield extrapolation based on Eq. 1 that uses both pressure and gradient data, from either monopole or dipole sources. It is noted that in this extrapolation step the “full model” used for extrapolation contains all scatterers/perturbations that generate scattering, as the gray-colored object indicates. The left side of FIG. 3 shows how the recorded time-reversed receiver-side gradient data (projected onto the normal nᵣ, e.g., FIG. 1) is injected at the model boundary as pressure boundary values. On the right side of FIG. 3, time-reversed, recorded pressure data (right panel), is injected as dipole (e.g, particle velocity) boundary values oriented according to nᵣ at each surface point. In both cases, the output receiver wavefields are stored as pressure responses. Finally, the total subsurface receiver wavefields G(x,xₛ) are obtained by subtracting the fields in the right side from their counterparts on the right side, I accordance with Eq. 1. The short red line in FIG. 3 represents the “minus sign” of Eq. 1. Two lines of formulae are shown in FIG. 3; the top line of formulae comprise formulae for a monopole source, discussed here, while the bottom line of formulae are formulae for a dipole source, discussed later on in this Description.

FIG. 4 illustrates, in accordance with an aspect of the present invention, Step 6 of the method 1100, above. It is noted that in this extrapolation step the “reference model” used for extrapolation does not contain all scatterers/perturbations that generate scattering. The gray-colored scatterers are not present in this reference model. Other than the scatterers, FIG. 4 is the same as FIG. 3. The left side of FIG. 4 shows how the recorded time-reversed receiver-side gradient data (projected onto the normal nᵣ, e.g., FIG. 1) is injected at the model boundary as pressure boundary values. The right hand side of FIG. 4, shows time-reversed, recorded pressure data (right panel) injected as dipole (e.g, particle velocity) boundary values oriented according to nᵣ at each surface point. In both cases, the output receiver wavefields are stored as pressure responses. Finally, the total subsurface receiver wavefields G₀(x,xₛ) is obtained by subtracting the fields on the right hand side from their counterparts on the left hand side of the figure, I accordance with Eq. 1. Again, the short red line in the figure represents the minus sign present in Eq 1. Similar to FIG. 3, two lines of formulae are shown, where the top line of formulae are formulae for a monopole source while the, bottom line formulae are formulae for a dipole source.

It is noted that the extrapolation method in accordance with an embodiment of the present invention described above may provide for the following:
The method jointly incorporates directional, amplitude and phase information from both pressure and gradient data in an exact manner, thus suitable for post-extrapolation “true amplitude” processing or imaging;
The method requires the receiver surface to enclose only the source location and not the subsurface targets;
The method is exact regardless of what the properties of the medium may be outside the receiver domain, thus being equally exact and applicable in cases where the subsurface target properties are arbitrarily heterogeneous, anisotropic, elastic and attenuative.

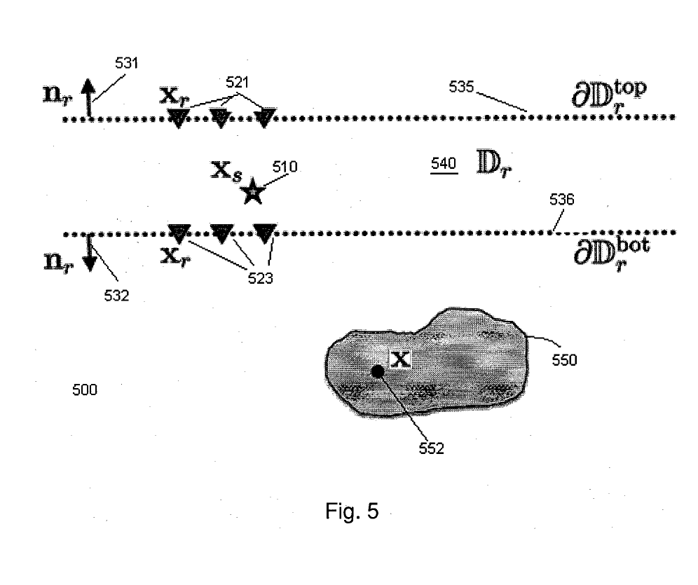

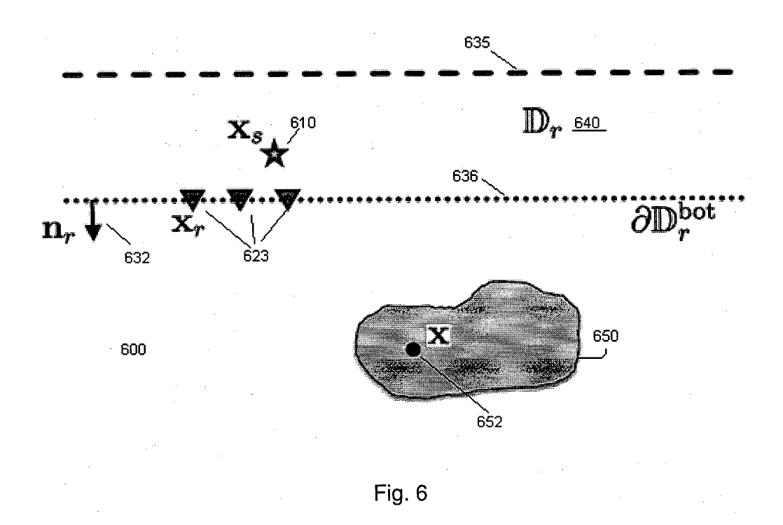

Having receivers on a 3D boundary that completely encloses the sources, as described in FIG. 1 and Eq. 1, is not always convenient or practical. Other survey geometries are possible. Two examples of other geometries are shown in FIGS. 5 and 6, which are based on planar receiver acquisition surfaces. Corresponding to the change in configuration of the sources from that of FIG. 1 to that of FIG. 5 or 6, Equation 1 changes into Equations 2 or 3.

$$\begin{matrix}{G}_{S} ( x , {x}_{s} ) = {\int }_{\partial {D}_{r}^{top} ⋃ \partial {D}_{r}^{bot}} \frac{- 1}{i ω ρ} [ {∇}_{{x}_{r}} {G}^{*} ( {x}_{r} , {x}_{s} ) {G}_{S} ( x , {x}_{r} ) - {G}^{*} ( {x}_{r} , {x}_{s} ) {∇}_{{x}_{r}} {G}_{S} ( x , {x}_{r} ) ] \cdot {n}_{r} {d}^{2} {x}_{r} & Equation 2 \\ {G}_{S} ( x , {x}_{s} ) = {\int }_{{D}_{r}^{bot}} \frac{- 1}{i ω ρ} [ {∇}_{{x}_{r}} {G}^{*} ( {x}_{r} , {x}_{s} ) {G}_{S} ( x , {x}_{r} ) - {G}^{*} ( {x}_{r} , {x}_{s} ) {∇}_{{x}_{r}} {G}_{S} ( x , {x}_{r} ) ] \cdot {n}_{r} {d}^{2} {x}_{r} & Equation 3\end{matrix}$$

In the configuration 500 as shown in FIG. 5 and Eq. 2, the one finite enclosed surface boundary ∂Dᵣ 130, as also shown in FIG. 1, becomes two “infinite” horizontal planes: ∂Dᵣ₋ₜₒₚ, a top plane 535 and ∂D_{r-bottom, a} bottom plane 536 that enclose the sources 510. The receivers 521; 523 are located on both top plane and bottom plane and enclose all sources 510 in the volume Dᵣ 540. When the boundary planes 535 and 536 are infinitely large, the configuration in FIG. 5 is equivalent to the configuration in FIG. 1. Correspondingly, for Eq. 2 the result is to integrate along the two planes. It is noted that the horizontal planes 535 and 536 of the receivers 521 and 523 are “enclosing” to the sources 510, not the subsurface structures 550 that are to be explored/investigated. Merely by way of example, in practice, the distance between the receiver planes 535 and 536 (which may be of the order of meters or 10s of meters and the sources are in between the receiver planes 535 and 536) is substantially smaller than the horizontal crossline or inline extent of the receivers (which may be of the order of kilometers or the like).

In the configuration 600, as shown FIG. 6 and Eq. 3, the top plane 535, as in FIG. 5, becomes a free surface 635, so no receivers are needed. As a result, there is only one plane, ∂D_{r-bottom}, the bottom plane 636. In this situation, the receivers 623 are located on the bottom plane 636. All sources 610 in the volume Dᵣ 640 are enclosed by the free surface 635 and the bottom plane 636. Correspondingly, the result for Eq. 3 is to integrate along the bottom plane 636.

Both configurations 500 and 600 in FIGS. 5 and 6 are special forms of the configuration 100 as in FIG. 1, so they share the same aspects as the method described above based on FIG. 1, as long as the receiver acquisition planes are large enough to be considered “infinite” within the scale of the experiment or “enclosing” the sources. The method can be applied to the configurations in depicted FIG. 5 and FIG. 6 with no modification. The free-surface configuration in FIG. 6 and Eq 3 may be directly implemented for marine seismic acquisition systems.

In the case of data acquired using physical dipole sources, a dipole subsurface extrapolated scattered field, analogous to that in Eq 1, can be obtained from

$${∇}_{{x}_{s}} {G}_{S} ( x , {x}_{s} ) = {∮}_{\partial {D}_{r}} \frac{- 1}{i ω ρ} [ {∇}_{{x}_{s}} {∇}_{{x}_{r}} {G}^{*} ( {x}_{r} , {x}_{s} ) {G}_{S} ( x , {x}_{r} ) - {∇}_{{x}_{s}} {G}^{*} ( {x}_{r} , {x}_{s} ) {∇}_{{x}_{r}} {G}_{S} ( x , {x}_{r} ) ] \cdot {n}_{r} {d}^{2} {x}_{r} \qquad Equation 4$$

The extrapolators in Eq. 4 are the same as those used for the monopole-source fields in Eqs 1-3, therefore, the wavefield extrapolation method for dipole-source data is the same as described above. In FIGS. 3-4, the bottom formulas also illustrate extrapolation of dipole-source data. The method for extrapolation of dipole-source data shares the same benefits as those outlined above for monopole-source data. Also, the configurations in FIGS. 5-6 are equally applicable to dipole-source data.

The equations and methodologies described above are based on correlation-type reciprocity. Other methods, in accordance with embodiments of the present invention, may be designed using one- and two-sided scattering reciprocity of the convolution type, which yields the identity:

$$- {G}_{S} ( x , {x}_{s} ) = {∮}_{\partial {D}_{r}} \frac{1}{i ω ρ} [ G ( {x}_{r} , {x}_{s} ) {∇}_{{x}_{r}} {G}_{S} ( x , {x}_{r} ) - {∇}_{{x}_{r}} G ( {x}_{r} , {x}_{s} ) {G}_{S} ( x , {x}_{r} ) ] \cdot {n}_{r} {d}^{2} {x}_{r} \qquad Equation 5 a$$

which is a convolution-type method, as opposed to the correlation-type extrapolation integrals in Eqs 1-3. This convolution-type extrapolation integral provides all of the same benefits of the correlation approaches above, and is also applicable to the cases in FIGS. 5-6. Eq. 5a is an equation for a monopole source. However, it may be extended to dipole-source data analogously to Eq 4, which is straight forward as provided in Eq. 5b:

$$- {∇}_{{x}_{s}} {G}_{S} ( x , {x}_{s} ) = {∮}_{\partial {D}_{r}} \frac{1}{i ω ρ} [ \begin{matrix}{∇}_{{x}_{s}} G ( {x}_{r} , {x}_{s} ) {∇}_{{x}_{r}} {G}_{S} ( x , {x}_{r} ) - \\ {∇}_{{x}_{s}} {∇}_{{x}_{r}} G ( {x}_{r} , {x}_{s} ) {G}_{S} ( x , {x}_{r} )\end{matrix} ] \cdot {n}_{r} {d}^{2} {x}_{r} \qquad Equation 5 b$$

The correlation-type method described above is easily modified to conduct convolution-type extrapolation 1200 in accordance with an embodiment of the present invention as follows::
(1) start with both pressure and gradient data on the receiver side (see FIG. 2) (1210);
(2) start with two separate, a priori, known models for subsurface proprieties, one of the models should contain all known heterogeneities/scatterers/perturbations (hereafter referred to as the “full model”), while the other model does not contain heterogeneities/scatterers/perturbations (hereafter referred to as the “reference model”) that account for the desired scattered field G_{S}(1220);
(3) no time reversal required (step may be skipped); weight data individually at receiver locations by the known (iωρ(xᵣ))⁻¹ factor prior to wavefield injection or convolution with extrapolators, project pressure and gradient data to local receiver normal direction nᵣ(xᵣ) according to Eq 5 (1230);
(4) jointly extrapolate pressure and gradient receiver data (as acquired, no time reversal required) using the full model according to FIG. 3, by simultaneously using data from all receivers (this implicitly evaluates the receiver integral): this yields the subsurface field G(x,xᵣ) at all x locations within the subsurface model (1240);
(5) jointly extrapolate pressure and gradient receiver data (as acquired, no time reversal required) using the reference model according to FIG. 4, by simultaneously using data from all receivers (this implicitly evaluates the receiver integral): this yields the subsurface field G₀(x,xᵣ) at all x locations within the subsurface model (1250); and
(6) obtain the subsurface extrapolated field G_{S}(x,xᵣ) from Eq 5 by subtracting the result from Step 5—(1250) from that of Step 4—(1240); the minus sign in Eq 5 is implicitly accounted for in the order of the subtraction terms in FIGS. 3-4 (1260).

The convolution extrapolation is equivalent to the correlation extrapolation, with only minor modifications, e.g. no time reversal is required, as noted above.

### EXAMPLE

### Marine Acquisition

As mentioned above, the geometry configuration 600 in FIG. 6 above is particularly suitable for acquisition of marine seismic data. That geometry is hereby used to describe the following marine acquisition geometries.

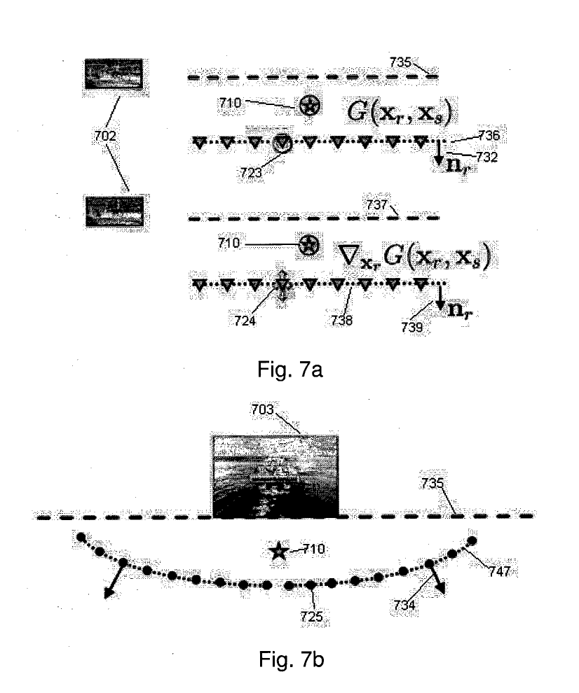

Referring to FIG. 7a, sources 710 and streamers 723, 724 are towed behind a vessel 702. The streamers are deep-towed, i.e. they are towed deeper than the sources 710 such that the receivers 723 and 724 in the streamers are located between the sources 710 and the sea floor 736 or 738, according to the configuration as in FIG. 6. For inline marine geometries: one or more consecutive or simultaneous seismic sources 710 (of monopole- or dipole-type) are positioned between water 735/737 and deep-towed streamers equipped with co-located pressure and gradient sensors 723/724; for crossline marine geometries: streamers equipped with co-located pressure and gradient sensors 723/724, deep-towed by one or more parallel-sailing seismic vessels in any configuration (as long as streamers are towed deeper than sources), recording the signal from one or more, consecutive or simultaneous seismic sources (of monopole- or dipole-type). The upper diagram shows monopole source 710 (e.g. airgun) with monopole sensors 723 (e.g. pressure sensors or hydrophones); the lower diagram shows monopole source 710 (e.g. airgun) with dipole/gradient sensors 724 measuring the vertical component of the gradient. The sensors are shown separately for clarity purpose. They are actually co-located on the same streamers.

In this example in FIG. 7a, the boundary 736/738 is the streamer plane. The normal direction (732/739) of the boundary (736/738) is simply the downward vertical direction. Merely by way of example, in one embodiment of the present invention, the measurements used to implement the extrapolation methods discussed earlier for marine seismic data acquisition are: (1) pressure; and (2) the vertical component of pressure gradient, which is equivalent to a measurement of the vertical component of particle velocity.

The same configuration as in FIG. 7a can also be used in an Ocean Bottom Cables (“OBC”) marine seismic survey, where the OBC replace the streamers, as discussed above.

One simple variation of configuration shown in FIGS. 6 and 7a is to arrange the receivers in a bowl shape 747. The receiver-bowl 747 and the free-surface top 735 form a completed enclosing boundary that encloses all sources 710. FIG. 7b shows a cross-section of a streamer array of one example in the crossline direction. The streamers are towed at different depths: the streamers towed near the center (or the source) are towed deeper while the streamers near the outer sides are towed shallower. The configuration can greatly reduce the horizontal extent of the receiver plane (or streamers) which in turn can reduce the cost of data acquisition and the subsequent data processing. Similar arrangement can be done along the inline direction.

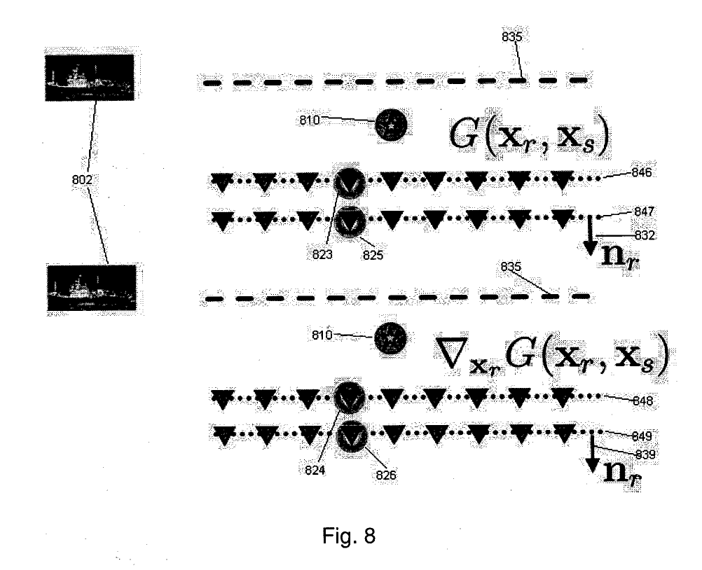

Referring to FIG. 8, in many towed marine seismic surveys, over/under streamers are useful. Related to the method describe here, rather than having multi-component streamers containing different sensors, e.g. both pressure and pressure gradient sensors, over/under streamers need only be traditional pressure sensors (i.e. hydrophones or the like). The measurement (i.e. pressure) is obtained from either the over 846/848 or under 847/849 streamer. The gradient (i.e. pressure gradient in this example) is obtained from differences of the two adjacent over/under (846/847 and 848/849) streamers. For inline marine geometries: one or more consecutive or simultaneous seismic sources 810 (of monopole- or dipole-type) are positioned between water 835 and deep-towed over/under streamers equipped with pressure sensors 823/824/825/826; for crossline marine geometries: over/under streamers equipped pressure sensors, deep-towed by one or more parallel-sailing seismic vessels 802 in any configuration (as long as streamers are towed deeper than sources), recording the signal from one or more consecutive or simultaneous seismic sources (of monopole- or dipole-type).

The acquisition geometries shown in FIGS. 7a, 7b and 8, or their variations, can be employed in any marine seismic survey, including narrow or wide azimuth acquisition, coil shooting and revolution survey acquisition technology. Once the data are acquired from geometries as shown in FIG. 7a, 7b or 8, the data can be processed to derive the exact extrapolated wavefield of subsurface, which can be used for many subsequent processes or investigations, one of which is further discussed below.

As explained above, the methods for data acquisition or extrapolation are applicable to any industries where wave phenomenon is involved. The examples related to marine seismic given above are for illustrative purposes and are not to limit the application of the methods. For example, the acquisition system 100 illustrated in FIG. 1 may be viewed as an onshore seismic data acquisition system. When the system 100 is interpreted as a top view of an onshore survey, the system 100 may be used to acquire or extrapolate surface wave properties (e.g. ground roll) of an area 150 outside the measurement boundary 130, where sources 110 or receivers 120 are located. The receivers 120 may measure one of the components of a wave (e.g. a vertical particle velocity) and one of its spatial gradients (e.g. a horizontal gradient of the vertical particle velocity where the gradient is normal to the boundary 130). The receivers 120 may measure many other wave properties (e.g. pressures, displacements, accelerations). Similarly, any of the acquisition systems illustrated in FIGS. 1-8 may be viewed as data acquisition systems for other industries, such as in CSEM, biomedical imaging, non-destructive remote sensing, underwater acoustic monitoring, space architecture design and engineering etc.

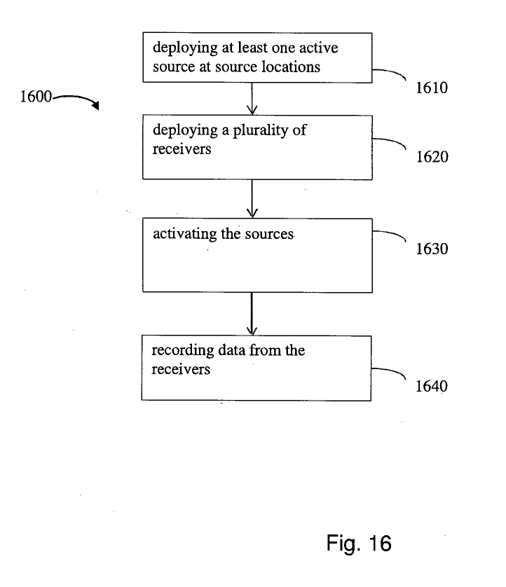

Methods for data acquisition, in accordance with embodiments of the present invention, which may be used in any industry for wavefield interpolation, may be summarized in a flow diagram as illustrated in FIG. 16, wherein the method 1600 may be performed as:
(1) deploying at least one active source 110 at source locations 110 (1610);
(2) deploying a plurality of receivers 120 at receiver locations 120 (1620), where the receivers 120 measure a wavefield quantity (e.g. a pressure or the like) and a component of its gradient (e.g. a vertical component of the pressure gradient or the like) that is normal (132) to a boundary 130 of measurement volume 140;
(3) activating source 110 (1630); and
(4) recording data from the receivers 120 (1640).
The acquired data are suitable for extrapolating wavefield properties within an unknown object (150) outside the boundary 130 of the measurement volume 140.

### Evaluation

The embodiments above provide methods that allow the extrapolation of exact data that otherwise would not be available. During the extrapolation, the methods require one or two models, and the quality of the models affect the resulting extrapolated data. It is noted that there are identities that can be used to measure the quality of the models, whether or not they are used in the methods described above. Similarly, there are related identities that can be used to measure the quality of the resulting extrapolated data.

Combining both correlation- and convolution-type scattering reciprocity from both the one- and two-sided formulations yields, in accordance with aspects of the present invention, the following identities:

$$\begin{matrix}0 = {ℐ}_{1} = {∮}_{\partial {D}_{r}} \frac{1}{i ω ρ} [ \begin{matrix}ℜ \{ G ( {x}_{r} , {x}_{s} ) \} {∇}_{{x}_{r}} {G}_{S} ( x , {x}_{r} ) - \\ ℜ \{ {∇}_{{x}_{r}} G ( {x}_{r} , {x}_{s} ) \} {G}_{S} ( x , {x}_{r} )\end{matrix} ] \cdot {n}_{r} {d}^{2} {x}_{r} & Equation 6 \\ 0 = {ℐ}_{2} = - {∮}_{\partial {D}_{r}} \frac{1}{i ω ρ} [ \begin{matrix}{G}_{S} ( {x}_{r} , {x}_{s} ) ℜ \{ {∇}_{{x}_{r}} {G}_{0} ( x , {x}_{r} ) \} - \\ {∇}_{{x}_{r}} {G}_{S} ( {x}_{r} , {x}_{s} ) ℜ \{ {G}_{0} ( x , {x}_{r} ) \}\end{matrix} ] \cdot {n}_{r} {d}^{2} {x}_{r} & Equation 7\end{matrix}$$

Both integrals are exact, can be applied in all configurations discussed above, and will hold for arbitrary medium properties away from the receiver domain (e.g., heterogeneity, anisotropy, elasticity, attenuation).

It is noted that these integrals have different sensitivity in terms of both the acquired data and the models used to derive extrapolators: Eq 6 uses the full data G(xᵣ,xₛ) and both the reference and full models to compute the extrapolator G_{S}(x,xᵣ), while Eq 7 relies only on the scattered portion of the observed data, i.e. G_{S}(xᵣ,xₛ), and on the reference model for the extrapolator G₀(x,xₛ).

Since the data in seismic surveys are a function of/are affected by the real subsurface properties, Eqs 6 and 7 will only hold when the extrapolators, i.e., the Earth model parameters, are “correct” and thus consistent with the recorded data. This in turn implies that by evaluating the integrals in Eqs 6-7 and measuring their deviations from zero, estimates of how acceptable the current Earth models are for the purpose of our exact extrapolation methods will be yielded. Although Eqs 6 and 7 are discussed here in relation to the exact extrapolation methods, they can also be used just for evaluating the quality of the subsurface model (without any extrapolation). When extrapolation is not used, the modeled object (e.g. 150 in FIG. 1) does not need to be outside the measurement boundary (e.g. 130 in FIG. 1) as discussed above. The target object (e.g. 150) may be inside or outside the boundary (e.g. 130) or partially inside and partially outside. The reference model may have scatterer information and spatially varying model parameters (e.g. P-wave velocity). The reference model does not need to have all parameters known.

A method 1400, in accordance with an embodiment of the present invention, to practically evaluate Eq. 6 using both models can be described as:
(1) perform the exact correlation-based wavefield extrapolation following the steps described using Eq 1 above, or method 1100 (1410);
(2) perform the exact convolution-based wavefield extrapolation following the steps described using Eq 5 above, or method 1200 (1420); and
(3) obtain identity (I1) by subtracting the result of Step 2—from that of Step 1—(1430).

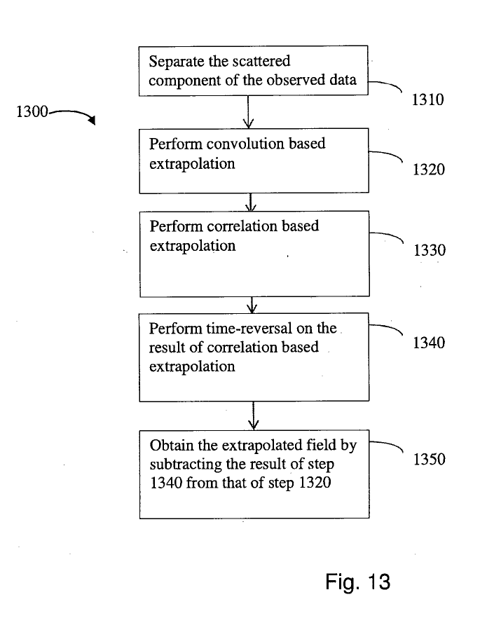

A method 1300, in accordance with an embodiment of the present invention, as in FIG. 13, to practically evaluate Eq 7 can be described as:
(1) using a priori information about the data and/or the model, separate the scattered/perturbed component of the observed data G_{S}(xᵣ,xₛ) (1310);
(2) perform the exact convolution-based wavefield extrapolation following the steps described using Eq 5 above, ensuring that only the scattered observed fields G_{S}(xᵣ,xₛ) are used instead of the full data G(xᵣ,xₛ), and that only the extrapolation in the reference model (FIG. 4) is carried out (1320);
(3) perform the exact correlation-based wavefield extrapolation following the steps described using Eq 1 above, ensuring that only the scattered observed fields G_{S}(xᵣ,xₛ) are used instead of the full data G(xᵣ,xₛ), and that only the extrapolation in the reference model (FIG. 4) is carried out (1330);
(4) perform time-reversal (in the time-domain) or take the complex conjugate (in frequency domain) of the extrapolated fields that result from step 3—(1340); and
(5) subtract the result of step 4 (1340) from that of step 2 (1320) (1350).

Once Eqs 6-7 are evaluated, one possible measure for model inaccuracy, in accordance with an embodiment of the present invention, is given by:

[symbol]

(m,x)=∫∥ [symbol] ₁(m,x)∥²d²xₛ+∫∥ [symbol] ₂(m,x)∥²d²xₛ Equation 8

where m is a vector with current known model parameters with samples at an x subsurface location. It is noted that:
this measure may vary at an x location, function of the model at x itself but also at possibly at all other subsurface locations;
this measure depends on both Eq 6 and Eq 7, but these are independent measures of model accuracy and can be used jointly as in Eq 8 or with arbitrary weights, or separately as independent measures;
although Eq 8 is in the frequency domain, the measure is equally valid in the time-domain, or can be jointly evaluated in both domains;
although Eq 8 employs the so-called L2 norm (or power norm) any other norm would yield an admissible measure for model inaccuracy;
although Eq 8 employs a summation over all available sources in the seismic experiment, estimates of model inaccuracy using Eqs 6-7 can also be conducted on a separate shot-by-shot basis;
using Eqs 6-7 for estimates of model inaccuracy, model inaccuracy estimates using the measure in Eq 8 or under any of the modifications in the items above is generally valid under all geometries and experiment configurations presented in this disclosure.

A method 1500 to evaluate Eq 8, in accordance with an embodiment of the present invention, can be described as follows:
(1) evaluate Eq 6 according to method above, compute local power spectra at an x location in the model domain (1510);
(2) stack result of step 1—over all available shots in the seismic experiment (1520);
(3) evaluate Eq 7 according to method above, compute local power spectra at an x location in the model domain (1530);
(4) stack result of step 3—over all available shots in the seismic experiment (1540); and
(5) sum the results from steps 2—and 4—(1550).

As discussed above, there are many aspects of embodiments of the present invention, including:
a correlation-based (reverse time) method for exact, nonlinear extrapolation of subsurface receiver wavefields that uses both pressure and gradient data, from only monopole sources;
a correlation-based (reverse time) method for exact, nonlinear extrapolation of subsurface receiver wavefields that uses both pressure and gradient data, from only dipole sources;
a correlation-based (reverse time) method for exact, nonlinear extrapolation of subsurface receiver wavefields that uses both pressure and gradient data, from both pressure and dipole sources;
a convolution-based (forward time) method for exact, nonlinear extrapolation of subsurface receiver wavefields that uses both pressure and gradient data, from only pressure sources;
a convolution-based (forward time) method for exact, nonlinear extrapolation of subsurface receiver wavefields that uses both pressure and gradient data, from only dipole sources;
a convolution-based (forward time) method for exact, nonlinear extrapolation of subsurface receiver wavefields that uses both pressure and gradient data, from both pressure and dipole sources;
two independent evaluation methods of measuring errors in subsurface Earth models, whether the models are used for extrapolation mentioned above or not, where the two evaluation methods can be used for parameter estimation jointly or separately; and
methods for acquiring marine vector-acoustic seismic data suitable for extrapolation.

In the above discussion where two models (a full model and a reference model) are used, the discussion and the use of extrapolation methods are associated with a single seismic survey or the like. The same extrapolation methods can be used when two or more surveys are involved, e.g. a time-lapse survey and an original survey. The scatterers in a single survey are the features or singularities of the subsurface that one is looking for, whose wavefield is extrapolated using the methods discussed here. In the context of a time-lapse survey, the scatterers are the perturbations or changes between the time-lapse survey and the original survey, whose wavefield is desired and which can be extrapolated using the same methods discussed here. The physical interpretation of scatterers or perturbations may be different, the mathematical representations and their wavefield extrapolation methods are the same. All the methods described here are applicable to time-lapse surveys. In this application, the scatterers and the perturbations are interchangeable, depending on the context where the methods are applied.

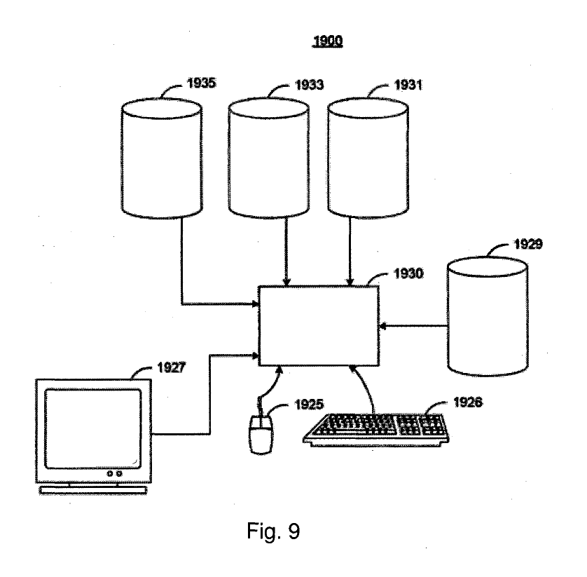

The data processing portions of the methods described above may be implemented in a computer system 1900, one of which is shown in FIG. 9. The system computer 1930 may be in communication with disk storage devices 1929, 1931, 1933 and 1935, which may be external hard disk storage devices. It is contemplated that disk storage devices 1929, 1931, 1933 and 1935 are conventional hard disk drives, and as such, will be implemented by way of a local area network or by remote access. Of course, while disk storage devices are illustrated as separate devices, a single disk storage device may be used to store any and all of the program instructions, measurement data, and results as desired.

In one implementation, data from the receivers may be stored in disk storage device 1931. Various data from different sources may be stored in disk storage device 1933. The system computer 1930 may retrieve the appropriate data from the disk storage devices 1931 or 1933 to process data according to program instructions that correspond to implementations of various techniques described herein. The program instructions may be written in a computer programming language, such as C++, Java and the like. The program instructions may be stored in a computer-readable medium, such as program disk storage device 1935. Such computer-readable media may include computer storage media. Computer storage media may include volatile and non-volatile, and removable and non-removable media implemented in any method or technology for storage of information, such as computer-readable instructions, data structures, program modules or other data. Computer storage media may further include RAM, ROM, erasable programmable read-only memory (EPROM), electrically erasable programmable read-only memory (EEPROM), flash memory or other solid state memory technology, CD-ROM, digital versatile disks (DVD), or other optical storage, magnetic cassettes, magnetic tape, magnetic disk storage or other magnetic storage devices, or any other medium which can be used to store the desired information and which can be accessed by the system computer 1930. Combinations of any of the above may also be included within the scope of computer readable media.

In one implementation, the system computer 1930 may present output primarily onto graphics display 1927, or via printer 1928 (not shown). The system computer 1930 may store the results of the methods described above on disk storage 1929, for later use and further analysis. The keyboard 1926 and the pointing device (e.g., a mouse, trackball, or the like) 1925 may be provided with the system computer 1930 to enable interactive operation.

The system computer 1930 may be located at a data center remote from an exploration field. The system computer 1930 may be in communication with equipment on site to receive data of various measurements. The system computer 1930 may also be located on site in a field to provide faster feedback and guidance for the field operation. Such data, after conventional formatting and other initial processing, may be stored by the system computer 1930 as digital data in the disk storage 1931 or 1933 for subsequent retrieval and processing in the manner described above. While FIG. 9 illustrates the disk storage, e.g. 1931 as directly connected to the system computer 1930, it is also contemplated that the disk storage device may be accessible through a local area network or by remote access. Furthermore, while disk storage devices 1929, 1931 are illustrated as separate devices for storing input seismic data and analysis results, the disk storage devices 1929, 1931 may be implemented within a single disk drive (either together with or separately from program disk storage device 1933), or in any other conventional manner as will be fully understood by one of skill in the art having reference to this specification.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. §112, paragraph 6 for any limitations of any of the claims herein, except for those in which the claim expressly uses the words ‘means for’ together with an associated function.

What is claimed is:

1. A method (1000) for extrapolating wavefield (152) data within an unknown object (150) where the unknown object (150) is located outside of a boundary (130) of a measurement volume (140), wherein at least one source (110) is located within the measurement volume (140) and a plurality of receivers (120) are used in the measurement volume (140) to measure a wavefield quantity and a component of a gradient of the wavefield quantity, where the component of the gradient of the wavefield quantity is normal (132) to the boundary (130) of the measurement volume (140), the method comprising:
using a first model to derive derived data of a wavefield quantity and a component of its gradient at one or more receiver locations, wherein the receiver locations are on the boundary (130) and wherein the component is normal to the boundary of the measurement volume (1030) at the receiver location (120); and
jointly extrapolating the wavefield data within the unknown object (150) using the measured data and the derived data according to a reciprocity scattering relation (1040).

2. The extrapolation method of claim 1, wherein the reciprocity scattering relation is in the form of a correlation or a convolution.

3. The extrapolation method of claim 1, wherein the source is a monopole source.

4. The extrapolation method of claim 1, wherein the source is a dipole source.

5. The extrapolation method of claim 1, wherein the plurality of receivers comprise monopole receivers.

6. The extrapolation method of claim 1, wherein the plurality of receivers comprise dipole receivers.

7. The extrapolation method of claim 3, wherein the reciprocity scattering relation is in the form of the following correlation:
$${G}_{S} ( x , {x}_{s} ) = {∮}_{\partial {D}_{r}} \frac{- 1}{i ω ρ} [ {∇}_{{x}_{r}} {G}^{*} ( {x}_{r} , {x}_{s} ) {G}_{S} ( x , {x}_{r} ) - {G}^{*} ( {x}_{r} , {x}_{s} ) {∇}_{{x}_{r}} {G}_{S} ( x , {x}_{r} ) ] \cdot {n}_{r} {d}^{2} \text{?}$$
$$\text{?} \text{indicates text missing or illegible when filed}$$

8. The extrapolation method of claim 3, wherein the reciprocity scattering relation is in the form of the following convolution:
$$- {G}_{S} ( x , {x}_{s} ) = {∮}_{\partial {D}_{r}} \frac{1}{i ω ρ} [ G ( {x}_{r} , {x}_{s} ) {∇}_{{x}_{r}} {G}_{S} ( x , {x}_{r} ) - {∇}_{{x}_{r}} G ( {x}_{r} , {x}_{s} ) {G}_{S} ( x , {x}_{r} ) ] \cdot {n}_{r} {d}^{2} \text{?}$$
$$\text{?} \text{indicates text missing or illegible when filed}$$

9. The extrapolation method of claim 4, wherein the reciprocity scattering relation is in the form of the following correlation:
$${∇}_{{x}_{s}} {G}_{S} ( x , {x}_{s} ) = {∮}_{\partial {D}_{r}} \frac{- 1}{i ω ρ} [ {∇}_{{x}_{s}} {∇}_{{x}_{r}} {G}^{*} ( {x}_{r} , {x}_{s} ) {G}_{S} ( x , {x}_{r} ) - {∇}_{{x}_{s}} {G}^{*} ( {x}_{r} , {x}_{s} ) {∇}_{{x}_{r}} {G}_{S} ( x , {x}_{r} ) ] \cdot {n}_{r} {d}^{2} \text{?}$$
$$\text{?} \text{indicates text missing or illegible when filed}$$

10. The extrapolation method of claim 4, wherein the reciprocity scattering relation is in the form of the following convolution:
$$- {∇}_{{x}_{s}} {G}_{S} ( x , {x}_{s} ) = {∮}_{\partial {D}_{r}} \frac{1}{i ω ρ} [ \begin{matrix}{∇}_{{x}_{s}} G ( {x}_{r} , {x}_{s} ) {∇}_{{x}_{r}} {G}_{S} ( x , {x}_{r} ) - \\ {∇}_{{x}_{s}} {∇}_{{x}_{r}} G ( {x}_{r} , {x}_{s} ) {G}_{S} ( x , {x}_{r} )\end{matrix} ] \cdot {n}_{r} {d}^{2} \text{?}$$
$$\text{?} \text{indicates text missing or illegible when filed}$$

11. The extrapolation method of claim 1, wherein the first model comprises scatterers and the extrapolation method (1100) further comprises:
having a second model of the unknown object (150), wherein the second model includes a homogeneous property of the first model that contains no scatterers;
using the second model to compute derived reference data of a wavefield quantity and a component of its gradient at receiver locations that is normal to the boundary of the measurement volume (1130);
wherein jointly extrapolating the wavefield data, further comprises:
jointly extrapolating time-reversed derived data and its gradient using the first model by simultaneously using measured data from all receivers yielding a subsurface field G (1140);
jointly extrapolating time-reversed derived reference data and its gradient using the second model by simultaneously using measured data from all receivers yielding a subsurface field G₀(1150); and
obtaining an extrapolated field G_{S} by subtracting G₀ (115) from G (1160).

12. The extrapolation method of claim 1, wherein the first model comprises scatterers; and the extrapolation method (1200) further comprises:
computing derived reference data of a wavefield quantity and a component of its gradient at receiver locations, wherein the component is normal to the boundary of the measurement volume (1230), and wherein the step of jointly extrapolating the wavefield further comprises:
jointly extrapolating derived data and its gradient using the first model by simultaneously using measured data from all receivers yielding a subsurface field G (1240);
jointly extrapolating derived reference data and its gradient using the a second model by simultaneously using measured data from all receivers yielding a subsurface field G₀ (1250), the second model comprising a model of the unknown object containing a homogeneous property of the first model without scatterers (1220);
obtaining the extrapolated field G_{S} by subtracting G₀ (1250) from G (1260).

13. The extrapolation method of claim 1, wherein:
the extrapolated wavefield data comprises is data extrapolated for a seismic survey; or
the extrapolated wavefield data comprises data extrapolated from a time-lapse seismic survey; and the first model and the second model are models for the subsurface at an original survey and the time-lapse survey.

14. The extrapolation method of claim 1, wherein the extrapolated wavefield data is used for at least one of the following: Controlled Source Electromagnetic surveys; acoustic space architecture, design and engineering; biomedical imaging with ultrasound or electromagnetic radiation; underwater acoustic monitoring; and non-destructive engineering monitoring.

15. The extrapolation method of claim 1, wherein the extrapolating wavefield is performed in at least one of a time-domain and a frequency domain.

16. A method for evaluating the accuracy of a first physical model of an unknown object with unknown model parameters (150) outside and/or inside a boundary (130) of a measurement volume (140), wherein the first model contains scatterer information and spatially varying model parameters, wherein at least one source is within the measurement volume and a plurality of receivers measuring a wavefield quantity and a component of its gradient at receiver locations, wherein the component is normal (132) to the boundary (130) of the measurement volume (140), the evaluation method (1300) comprising:
receiving the measured data of the wavefield quantity and the component of its gradient at a receiver location, wherein the component is normal to the boundary of the measurement volume; and
the evaluation method (1300) is characterized by:
using the first model computing derived data of a wavefield quantity and a component of its gradient at receiver locations, wherein the component is normal to the boundary of the measurement volume (1310); and
performing a correlation-based wavefield extrapolation (1330) and a convolution-based wavefield extrapolation (1320) using the measured data and the derived data according to a reciprocity scattering relation;
performing time-reversal on the results (1330) of correlation based extrapolation (1340);
obtaining a first identity by subtracting the time-reversed result of correlation (1340) from the result of convolution (1320), wherein the value of the first identity indicates the accuracy of the model (1350).

17. The method of claim 16, the method further comprising:
(1) performing correlation-based wavefield extrapolation, wherein the correlation-based wavefield extrapolation comprises:
(a) jointly extrapolating time-reversed derived data and its gradient using the first model by simultaneously using measured data from all receivers yielding a subsurface field G (1140);
(b) jointly extrapolating time-reversed derived reference data and its gradient using the second model by simultaneously using measured data from all receivers yielding a subsurface field G₀ (1150); and
(c) obtaining the extrapolated field G_{S} by subtracting a result from step (b) G₀ (1150) from a result of step (a) G (1160);
(2) performing convolution-based wavefield extrapolation, wherein the convolution-based wavefield extrapolation comprises:
(d) jointly extrapolating derived data and its gradient using the first model by simultaneously using measured data from all receivers yielding a subsurface field G (1240);
(e) jointly extrapolating derived reference data and its gradient using the second model by simultaneously using measured data from all receivers yielding a subsurface field G₀ (1250); and
(f) obtaining the extrapolated field G_{S} by subtracting the result from step (e) G₀ (1250) from the result of step (d) G (1260);
(3) obtaining a second identity by subtracting the result of step (2) from the result of step (1); wherein the second identity indicates the accuracy of the model and the extrapolated wavefields (1430).

18. The evaluation method of claim 17, further comprising:
(1) evaluating a first identity and computing a local power spectra at an x location in the model domain (1510);
(2) stacking a result of step (1) over all available shots in the data (1520);
(3) evaluating a second identity and computing local power spectra at an x location in the model domain (1530);
(4) stacking a result of step (3) over all available shots in the data (1540); and
(5) summing results from steps (2) and (4) (1550).

19. The evaluation method of claim 17, wherein:
the identities are in the form of I₁, I₂ or J(m,x) as follows:
$$\begin{matrix} 0 = {ℐ}_{1} = {∮}_{\partial {D}_{r}} \frac{1}{i ω ρ} [ \begin{matrix}ℜ \{ G ( {x}_{r} , {x}_{s} ) \} {∇}_{{x}_{r}} {G}_{S} ( x , {x}_{r} ) - \\ ℜ \{ {∇}_{{x}_{r}} G ( {x}_{r} , {x}_{s} ) \} {G}_{S} ( x , {x}_{r} )\end{matrix} ] \cdot {n}_{r} \text{?} \\ 0 = {ℐ}_{2} = - {∮}_{\partial {D}_{r}} \frac{1}{i ω ρ} [ \begin{matrix}{G}_{S} ( {x}_{r} , {x}_{s} ) ℜ \{ {∇}_{{x}_{r}} {G}_{0} ( x , {x}_{r} ) \} - \\ {∇}_{{x}_{r}} {G}_{S} ( {x}_{r} , {x}_{s} ) ℜ \{ {G}_{0} ( x , {x}_{r} ) \}\end{matrix} ] \cdot {n}_{r} \text{?} , \\ or \\ 𝒥 ( m , x ) = \int {‖ {ℐ}_{1} ( m , x ) ‖}^{2} {d}^{2} {x}_{s} + \int {‖ {ℐ}_{2} ( m , x ) ‖}^{2} {d}^{2} {x}_{s} ; \\ \text{?} \text{indicates text missing or illegible when filed}\end{matrix}$$
and deviation from zero indicates a magnitude of inaccuracy.

20. The method of claim 1 is further characterized by using any methods in claims 16-19 to evaluating the accuracy of the model(s) or the extrapolated wavefield (1300, 1400, 1500).

21. A system processing data for extrapolating wavefield within an unknown object outside the boundary of a measurement volume, wherein at least one source is within the measurement volume and a plurality of receivers measuring a wavefield quantity and a component of its gradient at receiver locations that is normal to the boundary of the measurement volume, the system comprising:
a processor,
a computer readable storage medium storing computer executable instructions, wherein when executed by the processor, the processor is to perform any methods as in claims 1-20.

22. The method of claim 1, further comprising:
using the extrapolated wavefield measurement data within the unknown object to process an image of the Earth's interior.

23. The method of claim 1, further comprising:
using the extrapolated wavefield measurement data within the unknown object to process a medical image.
